(19) United States
(12) Patent Application Publication
(10) Pub. No.: US 2024/0117296 A1
(43) Pub. Date: Apr. 11, 2024

(54) NON-TOXIC ORGANIC PETROLEUM HYDROCARBONS AND ORGANIC COMPOUNDS DEGRADATION AND REMEDIATION FORMULATION AND METHODS OF USE THEREOF

(71) Applicant: EVERGREEN PRODUCTS, LLC, Kyle, TX (US)

(72) Inventor: Kristopher Williams, Kyle, TX (US)

(21) Appl. No.: 18/378,195

(22) Filed: Oct. 10, 2023

Related U.S. Application Data
(60) Provisional application No. 63/414,968, filed on Oct. 11, 2022.

(51) Int. Cl. C12N 1/20; B09C 1/10; C02F 3/34; C11D 3/38

(52) U.S. Cl. CPC C12N 1/20; B09C 1/10; C02F 3/341; C11D 3/381; C02F 2101/32

(57) ABSTRACT

The present application discloses a non-toxic organic formulation for decreasing remediation time for released petroleum products and/or organic compounds into soil and water and the degradation of organic compounds applied during industrial processes. The non-toxic organic formulation comprising consortium of microbes and enhancers that tend to digest the petroleum hydrocarbon products including but not limited to oils, refined fuels, and greases into non-toxic byproduct and degrade organic compounds including but not limited to acrylamide, benzene, toluene, ethylbenzene, and xylene into non-toxic byproducts.

### FIELD OF THE PRESENT INVENTION

The present application relates to the remediation of organic petroleum hydrocarbon and organic compound environmental contaminants from contaminated areas and the degradation of intentionally applied organic compounds. Organic compounds being any chemical compound that contains carbon-hydrogen, carbon-carbon, or any chemical compound in which one or more atoms of carbon are bonded to atoms of other elements. More particularly, the present application discloses a non-toxic organic formulation that digests petroleum hydrocarbons and organic compounds from areas contaminated therewith and degrades intentionally applied organic compounds. The present application also discloses a method for decreasing remediation time for released organic petroleum hydrocarbons and organic compounds into various areas and a method for cleaning such areas. The present application also discloses a method for degrading organic compounds that are intentionally applied during industrial processes.

### BACKGROUND OF THE DISCLOSURE

Crude oil is a naturally existing petroleum hydrocarbon which is further processed to form many products including organic compounds. The petroleum hydrocarbons may further include but are not limited to fuels, oils, greases, etc. and the organic compounds may include but are not limited to acrylamide, benzene, toluene, ethylbenzene, xylene, etc. The petroleum hydrocarbons and organic compounds are extensively used worldwide in form of a fuel, oil well fracturing chemicals, and other items for various utilities of human life. However, there may be accidental incidences which may lead to exposure of such hydrocarbons and organic compounds to environment, human, animal and plant life, resulting in damage thereto. For example, transportation, industrial, agricultural, and other sectors may lead to spilling off such petroleum hydrocarbons and organic compounds, or leakage thereof. Consequently, there may be severe damage to ecosystem and living beings. There may be instances where such products are merely dumped into soil or water bodies, thereby contaminating the soil and drinking water. The contaminated soil may further have severe consequences which may impact fertility thereof and quality and nutritional content of crops grown on such soil. Additionally, there are instances when petroleum hydrocarbons and organic compounds including acrylamide are intentionally applied to the environment during industrial processes.

There are many conventional processes and equipment which lead to remediation and degradation of such hydrocarbons and organic compounds released or applied into the areas. Such processes may be purely chemical based, physical, or biological. The biological processes which are environment friendly are being preferred and may involve microbes. For example, one of the conventional prior arts involves consortium of microbes along with some other components, for example emulsifier, nutritive component etc. Another conventional art involves biocatalyst mixture. Some of these issues are partially addressed by existing systems described below.

The patent application US 2021/0301191 A1, titled “Enhancing Oil and Gas Production through Compositions and Methods,” primarily focuses on improving the extraction of oil and gas from wells and associated equipment. Further, the composition includes a combination of chemical or synthetic solvents, surfactants, and biological components, with a specific focus on functions such as paraffin control, corrosion prevention, inhibition of bacterial growth, and the enhancement of oil recovery processes within the oil and gas industry.

Another US patent application US 2018/0056346 A1, titled “Methods for bioremediation of hydrocarbon-contaminated media” pertains to catering primarily to the specific demands and concerns of the oil and gas industry. Further, the disclosed method enhances production processes within the industry and tackle challenges unique associated with it. Further, the method relies on the deployment of concentrated acids and chemicals and is limited to the oil and gas sector only.

Yet another U.S. Pat. No. 11,097,140 B2, titled “Processes for remediation of a contaminated material” relies on bacterial microorganisms activated with nutrients as the key remediation method. The disclosed processes approach predominantly centers on the simplicity of applying the remediation formulation to contaminated materials.

Thus, every conventional art provides different composition having constituents in different proportion. Perhaps efficiency thereof may be measured only by comparing amount of time required to remediate a particular area and side effects caused thereby. However, the existing formulations may be toxic to nature and surroundings. The existing formulations may be inorganic and thus may be unsafe to the surroundings. Therefore, there exists a need for developing non-toxic organic formulations which may be completely safe to the surroundings and require less remediation time to get rid of petroleum hydrocarbons and organic compounds therefrom.

### BRIEF SUMMARY OF THE DISCLOSURE

In a first aspect, the present application discloses a non-toxic organic formulation. The non-toxic organic formulation comprising a consortium of microbial strains applied on dextrose, wheat bran, and/or a broth. The consortium of microbial strains includes microbes selected from the genus Bacillus and Pseudomonas including but not limited to Bacillus megaterium, Bacillus thuringiensis, Bacillus pumilus, Bacillus subtilis, Bacillus amyloliquefaciens, and Pseudomonas putida. The formulation also comprising a nitrogen-rich fertilizer, a phosphate-rich fertilizer, a surfactant, and a microbial inoculant. The microbial inoculant includes microbes selected from the genus Lactobacillus and actinomyces including but not limited to Lactobacillus Casei and Actinomycetes.

In a per gallon embodiment, the formulation comprising at least 2.2 trillion colony forming units or 0.55 lbs. of a consortium of microbial strains, at least 1.27 lbs of the nitrogen-rich fertilizer, at least 0.42 lbs. of the phosphate-rich fertilizer; at least 2.5 oz of the surfactant; and at least 1 oz of the microbial inoculant. This concentrate formulation can be increased in volume by increasing the formulation ingredients on a percentage by volume or percentage by weight basis indefinitely.

In the embodiment, the nitrogen-rich fertilizer is prilled urea fertilizer to provide pure nitrogen at concentration of 46% nitrogen. The phosphate-rich fertilizer is monoammonium phosphate fertilizer or a phosphoric acid fertilizer to provide pure phosphate at concentration of 61%. The surfactant is a liquid comprising sodium laureth sulfate, sodium lauryl sulfate and lauramine oxide.

In another embodiment, the formulation is in concentrated form. The formulation may be in diluted form by diluting the concentrated form of the formulation with water at 1:20 ratio.

As a second aspect, the present application discloses use of the non-toxic organic formulation for decreasing remediation time for released petroleum products and degrading organic compounds present in various areas and from various applications. The petroleum hydrocarbons including but not limited to oils, refined fuels, and greases and the organic compounds including but not limited to acrylamide, benzene, toluene, ethylbenzene, xylene, etc. The formulation has a tendency to digest and degrade the petroleum products and organic compounds into non-toxic byproducts.

In the embodiment, the formulation per gallon comprising at least 2.2 trillion colony forming units or 0.55 lbs. of a consortium of microbial strains, at least 1.27 lbs. of the nitrogen-rich fertilizer, at least 0.42 lbs. of the phosphate-rich fertilizer; at least 2.5 oz of the surfactant; and at least 1 oz of the microbial inoculant. This concentrate formulation can be increased in volume by increasing the formulation ingredients on a percentage by volume or percentage by weight basis indefinitely.

In a third aspect, a method for decreasing remediation time for released petroleum products and/or organic compounds present in an area is disclosed. The method includes providing an amount of the non-toxic organic formulation. Thereafter, the contaminated area is treated with the formulation. The treatment is provided to further decrease remediation time for the released petroleum products into the contaminated area.

In the embodiment, the method comprising applying the formulation in spray diluted form on the contaminated soil at a rate of at least 1 gallon per 50 square feet or 1 gallon per cubic yard. The method comprising mixing the formulation in diluted form with contaminated soil at a rate of at least 1 gallon per 50 square feet or 1 gallon per cubic yard.

In another embodiment, the method comprising mixing the formulation with contaminated water at a rate of at least 1 gallon per 50 gallons of water.

In a fourth aspect, a method for cleaning at least a portion of an area contaminated with petroleum products is disclosed. The method includes providing the non-toxic organic formulation which may be further applied on the area to remediate the contaminants including petroleum products and/or organic compounds.

In a fifth aspect, a method for degrading acrylamide and organic compounds present in petroleum product wells, fluids associated with petroleum product wells, or other industrial processes is disclosed. The method includes providing the non-toxic organic formulation which may be applied to the petroleum product wells, fluids associated with petroleum product wells, or other industrial processes. Thereafter, the petroleum product well, fluids associated with petroleum product well, or other industrial processes is treated with the formulation. The treatment is provided to degrade acrylamide including petroleum products and organic compounds.

### DETAILED DESCRIPTION OF THE EMBODIMENTS

For the purpose of promoting an understanding of the principles of the invention, reference will now be made to the exemplary embodiments, and specific language will be used to describe the same. It will nevertheless be understood that no limitation of the scope of the invention is thereby intended. Any alterations and further modifications of the inventive features described herein, and any additional applications of the principles of the invention as described herein, which would occur to one skilled in the relevant art and having possession of this disclosure, are to be considered within the scope of the invention.

The present application pertains to a non-toxic organic formulation which may have capacity to digest petroleum products and organic compounds released into an area or specifically applied during industrial processes. In some aspects, the formulation may have a tendency to decontaminate areas polluted with petroleum products and organic compounds. In some aspects, the formulation may have tendency to decrease remediation time for released the petroleum products and organic compounds into the areas. In some aspects, the formulation may have a tendency to degrade acrylamide or other organic compounds applied during industrial processes. In some aspects, the formulation may have tendency to increase degradation time of acrylamide and organic compounds applied during industrial processes. The areas may include such as but are not limited to natural or man-made surfaces above or below ground containing soil or water or both or neither. The petroleum products may include such as but are not limited to hydrocarbons selected from oils, refined fuels and greases. The organic compounds may include but are not limited to to acrylamide, benzene, toluene, ethylbenzene, xylene, etc.

The formulation is a natural solution which may include multiple microbes and soil enhancers. The microbes may be selected from the genus Bacillus and Pseudomonas including but not limited to Bacillus megaterium, Bacillus thuringiensis, Bacillus pumilus, Bacillus subtilis, Bacillus amyloliquefaciens, and Pseudomonas putida. The microbes are in spore form. Therefore, they may generally be spray applied to wheat bran or other carrier. However, as the wheat bran is insoluble, the consortium of microbes may be available as being sprayed on dextrose, broth, or other soluble carrier.

The soil enhancers may include such as but are not limited to a nitrogen-rich fertilizer and a phosphate-rich fertilizer. The nitrogen-rich fertilizer may be a prilled urea fertilizer. The prilled urea fertilizer may be provided provide pure nitrogen at concentration of 46%. The fertilizer may have the highest concentration of content of all the conventional solid nitrogenous fertilizers.

In the embodiment, the phosphate-rich fertilizer may be monoammonium phosphate fertilizer or phosphoric acid. The fertilizer may be formulated to provide pure phosphate at concentration of 61%. The formulation may provide the high concentration of the pure phosphate.

In the embodiment, the formulation may also include a surfactant. The surfactant may be a commercial grade dishwashing liquid. The commercial grade dishwashing liquid may act as surfactant during the spray application. The surfactant may include any other commercially available foaming agents which may further include such as but are not limited to sodium laureth sulfate, sodium lauryl sulfate and lauramine oxide.

The formulation may yet include a microbial inoculant which may be commercially available inoculant meant for soil. The microbial inoculant may be used for inhibiting the development of pathogenic microbes and improving native seed germination. The primary constituent of the inoculant—the Lactobacillus Casei may act as a preservative and probiotic to prevent unwanted strains from propagating during storage.

In some embodiments, the aforementioned formulation is a concentrate. The formulation may further be diluted with water at 1:20 ratio. The diluted formulation may be sprayed on contaminated soil at a rate of at least 1 gallon per 50 square feet or 1 gallon per cubic yard. In another embodiment, the diluted formulation may be mixed with contaminated soil at a rate of at least 1 gallon per 50 square feet or 1 gallon per cubic yard. The diluted formulation is mixed with contaminated water at a rate of at least 1 gallon per 50 gallons of water. Therefore, the formulation may tend to decrease remediation time for the released petroleum products and organic compounds into the soil or water or both. In some aspects, the present application discloses the non-toxic organic formulation to convert the petroleum products and organic compounds into non-toxic by-products. In some aspects, the formulation may tend to decrease remediation time for released petroleum products and organic compounds present in an area. In some aspects, the formulation may tend to decontaminate an area contaminated with petroleum products and organic compounds. In some aspects, the formulation may tend to degrade acrylamide applied during industrial processes including but not limited to those associated with oil and gas production.

In another embodiment, the present application discloses a method for decreasing remediation time for released petroleum products and organic compounds present in an area. The method may include a number of steps, sequence thereof may be exemplary to understand the present application. The method may include providing the predefined amount of the formulation, followed by applying thereof on the area. Methods of applying the formulation may include such as but not limited to spraying, coating, mixing, fumigating, and so on. The formulation may tend to clean the area contaminated with petroleum products and organic compounds.

In the application, unless specified otherwise, the terms “comprising”, “comprise”, and grammatical variants thereof, are intended to represent “open” or “inclusive” language such that they include recited elements but also permit the inclusion of additional, non-explicitly recited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. The description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The non-toxic organic formulation, a method for decreasing remediation time for released petroleum products present in an area, and a method for cleaning at least a portion of an area contaminated with petroleum products are disclosed. The present disclosure introduces an advancement to the field of petroleum hydrocarbon and organic compounds degradation by offering a range of technical advantages at least in terms of efficiency, effectiveness, cost effectiveness, transportability, versatility, safety, stability, environmental impact, compatibility, production, ease of user, flexibility, and scalability. The present disclosure effectively reduces the total time to degrade petroleum hydrocarbons and organic compounds, thereby improving the efficiency. The present disclosure effectively degrades petroleum hydrocarbons more readily with the additional ability to degrade organic compounds, thereby improving the effectiveness. The formulation of the present disclosure effectively is cheaper for the user to utilize to degrade petroleum hydrocarbons and organic compounds, thereby improving the cost effectiveness. Due to the patents specialized concentrated formula, the formulation may be more efficiently transported and stored until ready to be used, thereby improving transportability. Further, the formulation may be applied in a variety of ways to a variety of mediums per the needs or capabilities of the user, thereby improving versatility. Also, the formulation is non-toxic and safe for the natural environment, thereby reducing environmental impact. The formulation may safely be utilized in tandem with additional standard industry practices, thereby improving compatibility.

Further, the formulation can readily be produced with commercial grade products, thereby improving production. The formulation only requires the user to dilute the concentrate with water prior to application and requires no training or the development of new procedures, thereby improving ease of user. The present disclosure, due to its design, may be tailored to the user's specific requirements, saving money, and being more efficient, thereby improving the flexibility. Furthermore, the formulation has the ability to be produced in any volume that is required, thereby improving scalability.

Numerous embodiments of the invention are possible. The previous exemplary embodiments are intended to merely sake of understanding the invention, and not limit, the breadth and depth of embodiments that can fall within the scope of the appended claims and future claims, which define the invention. For example, the apparatus will be scaled to accommodate different flow rates of the water to be treated or impregnated. The chemical flow rates, hence, the concentration of the chemistry, and the pressure in the system may be adjusted depending on the contaminants to be treated and/or the particular application.

It will be apparent that various other modifications and adaptations of the application will be apparent to the person skilled in the art after reading the foregoing disclosure without departing from the spirit and scope of the application and it is intended that all such modifications and adaptations come within the scope of the appended claims.

What is claimed is:

1. A non-toxic organic formulation comprising:
a consortium of microbial strains applied on at least one of: wheat bran, dextrose, and broth, the consortium of microbial strains comprising:
Bacillus megaterium;
Bacillus thuringiensis;
Bacillus pumilus;
Bacillus subtilis;
Bacillus amyloliquefaciens;
Pseudomonas putida;
a nitrogen-rich fertilizer;
a phosphate-rich fertilizer;
a surfactant; and
a microbial inoculant comprising:
Lactobacillus Casei; and
Actinomycetes.

2. The non-toxic organic formulation of claim 1, further comprises at least 0.6 lbs. of consortium of microbial strains, at least 1.45 lbs. of the nitrogen-rich fertilizer, at least 0.5 lbs. of the phosphate-rich fertilizer; at least 10 oz of the surfactant; and at least 1 oz of the microbial inoculant.

3. The non-toxic organic formulation of claim 1, wherein the non-toxic organic formulation is at least one of: a concentrated formulations and a diluted formulation that is diluted with water at 1:20 ratio.

4. The non-toxic organic formulation of claim 3, wherein the diluted formulation is at least one of: spray applied with contaminated soil, mixed with contaminated soil, and mixed with contaminated water at a rate of at least 1 gallon per 50 square feet or 1 gallon per cubic yard.

5. The non-toxic organic formulation of claim 1, wherein the microbes of the consortium are in spore form and the non-toxic organic formulation comprises the consortium in spray form applied to wheat bran as a carrier.

6. The non-toxic organic formulation of claim 1,
wherein the nitrogen-rich fertilizer is prilled urea fertilizer to provide pure nitrogen at concentration of 46-0-0; and
wherein the phosphate-rich fertilizer is monoammonium phosphate fertilizer to provide pure phosphate at concentration of 12-61-0.

7. The non-toxic organic formulation of claim 1, wherein the surfactant is a dishwashing liquid comprising sodium laureth sulfate, sodium lauryl sulfate and lauramine oxide.

8. The non-toxic organic formulation of claim 1, wherein the Lactobacillus Casei is a probiotic and preservative to prevent unwanted microbial strains from propagating during storage.

9. The non-toxic organic formulation of claim 6, wherein the non-toxic organic formulation is comprising the consortium in spray form applied on dextran.

10. The non-toxic organic formulation of claim 1, wherein the non-toxic organic formulation decreases remediation time for released petroleum products present in a plurality of areas.

11. The non-toxic organic formulation of claim 10, wherein the petroleum products comprise at least one of: oils, fuels, and greases.

12. The non-toxic organic formulation of claim 10, wherein the non-toxic organic formulation digests the petroleum products into non-toxic byproducts.

13. The non-toxic organic formulation of claim 10, wherein the plurality of areas comprises at least one of: water and soil contaminated with the released petroleum products.

14. A method for decreasing remediation time for released petroleum products present in an area, the method comprising:
(a) providing an amount of a non-toxic organic formulation comprising:
a consortium of microbial strains applied at least one of: wheat bran, dextrose, and broth, the consortium of microbial strains comprising:
Bacillus megaterium;
Bacillus thuringiensis;
Bacillus pumilus;
Bacillus subtilis;
Bacillus amyloliquefaciens;
Pseudomonas putida;
a nitrogen-rich fertilizer;
a phosphate-rich fertilizer;
a surfactant; and
a microbial inoculant comprising:
Lactobacillus Casei; and
Actinomycetes; and
(b) treating at least a portion of the area contaminated with the petroleum products with the formulation, thereby decreasing remediation time for the released petroleum products thereinto.

15. The method of claim 14, wherein the contaminated area comprises at least one of:
soil and water.

16. The method of claim 14, wherein the method further comprises applying the formulation in spray diluted form on the contaminated soil at a rate of at least 1 gallon per 50 square feet or 1 gallon per cubic yard.

17. The method of claim 14, wherein the method further comprises mixing the formulation in diluted form with contaminated soil at a rate of at least 1 gallon per 50 square feet or 1 gallon per cubic yard.

18. The method of claim 14, wherein the method further comprises mixing the formulation with contaminated water at a rate of at least 1 gallon per 50 gallons of water.

19. A method for cleaning at least a portion of an area contaminated with petroleum products, the method comprising:
(a) providing an amount of a non-toxic organic formulation comprising:
a consortium of microbial strains applied on dextrose, the consortium of microbial strains comprising:
Bacillus megaterium
Bacillus thuringiensis
Bacillus pumilus
Bacillus subtilis
Bacillus amyloliquefaciens
Pseudomonas putida
a nitrogen-rich fertilizer;
a phosphate-rich fertilizer;
a surfactant; and
a microbial inoculant comprising:
Lactobacillus Casei; and
Actinomycetes; and
(b) applying the formulation to the area to get rid of the petroleum products.

20. The method of claim 19, wherein the method further comprises applying the formulation in spray diluted form on at least one of: contaminated soil, mixed with contaminated soil, and mixed with contaminated water at a rate of at least 1 gallon per 50 square feet or 1 gallon per cubic yard.
